(19) United States
(12) Patent Application Publication
(10) Pub. No.: US 2003/0087335 A1
(43) Pub. Date: May 8, 2003

(54) Process for extracting astaxanthin pigment from yeast and extracted pigment thereof

(76) Inventors: Ji Young Han, Seoul (KR); Seung Jae Lee, Puchon, Kyonggi-do (KR); Myung Kyo Jung, Ahnyang, Kyonggi-do (KR); Seok Keun Choi, Seoul (KR); Jung Seop Roh, Seoul (KR)

Correspondence Address: JACOBSON HOLMAN PLLC, 400 SEVENTH STREET N.W., SUITE 600, WASHINGTON, DC, 20004, US

(21) Appl. No.: 10/019,272

(22) PCT Filed: May 2, 2001
(86) PCT No.: PCT/KR01/00719

(30) Foreign Application Priority Data
May 4, 2000 (RU) 2000-23851

(51) Int. Cl. C12P 23/00; C11B 1/00

(52) U.S. Cl. 435/67; 554/8

(57) ABSTRACT

The present invention relates to a process for extracting astaxanthin pigment from the yeast cells of Phaffia rhodozyma comprising the steps of i) cultivating the yeast, ii) treating yeast culture suspension with microwave to destroy the cell walls and microbodies, iii) drying it or extracting astaxanthin pigment using the solvent selected from the group consisting of ethanol, methanol, acetone and mixture of them. Further, the present invention also relates to the astaxanthin extracted by above method.

### TECHNICAL FIELD

The present invention relates to a process for extracting astaxanthin pigment from the yeast cells of Phaffia rhodozyma comprising the steps of i) cultivating the yeast cells, ii) treating yeast culture suspension with microwave to destroy the cell walls and microbodies, iii) drying it or extracting astaxanthin pigment using the solvent selected from the group consisting of ethanol, methanol, acetone and mixture of them. Further, the present invention also relates to the astaxanthin extracted by above method.

More particularly, the present invention relates to a process for extracting astaxanthin pigment from the yeast culture suspension further comprising i) the microwave treatment step, in which the culture suspension is passed through the Teflon tube or is laid on the Teflon extraction vessel for irradiation of microwave 50˜1000 watts at the frequency of 900˜930 or 2400˜2500 MHz; ii) the pigment separation step, in which the obtained pigment is concentrated at reduced pressure using rotary vacuum evaporator. Further, the microwave treatment also provides the sterility of other microorganisms as well as the destruction of cell walls and microbodies in yeast.

### BACKGROUND ART

Astaxanthin (3,3′-dihydroxy-β,β′-carotene-4,4′-dione) is generally obtained from the yeast cell of Phaffia rhodozyma (Phytochenistry, 15, 1009, (1976)), blue-green algae of Haematococcus species (Phytochemistry, 20, 2561, (1981)) and Brevibacterium (J. general and applied microbiology, 15, 127, (1969)). Further, it is chiefly distributed in the sea water animals and fresh water animals (Phytochemistry 15. 1003-1007 (1976)).

Especially, it has been found in the Crustacea, such as shrimps or crawfishes ; the tips or yolks of birds ; trouts and salmons as scalet color (Critical Reviews in Biotechnology 11(4), 297-326(1991)). It contributes to the enhancement of color and flavor, the activation of immunity, the anticancer activity by removing oxygen free radical (Pure & Appl. Chem., 51. 649-660(1979), J. Korean. Soc. Food Sci. Nutr. 27(1). 163-167(1998)) and the anti-aging metabolism (Internat. J. Vit. Nutr. Res. 65. 79-86(1995)). Further, it can be used as a precursor of vitamin A.

On the other hand, it has been reported that the astaxanthin pigment has an excellent anti-oxidation activity compared to other carotenoid pigment or tocopherol (Fisheries Sci., 62. 134-140 (1996)). It has been regarded that the astaxanthin pigment has an importance of medical use as well as that of edible pigment (Crit. Rev. Biotechnol. 11. 297-326 (1991)).

However, the extraction of astaxanthin from the Crustacea, such as shrimps or crawfishes has been hardly tried, because only a small amount of astaxanthin is contained. Further, the extraction of astaxanthin from a Phaffia rhodozyma has been hardly applied either, since the cell walls of such microorganisms are too hard to extract astaxanthin from them.

In order to obtain the astaxanthin from the yeast cell of Phaffia rhodozyma, the development of efficient method for destroying the cell walls of Phaffia rhodozyma is necessarily required. Sometimes, to avoid the elevation of the cost, the dried astaxanthin powder without destroying the cell walls has been used (J. Applied phycology 4, 267-279(1992)). However, the astaxanthin powder manufactured without such treatment shows very low bioavailability, when it is used as animal feed or food additive.

Therefore, a number of methods for destroying cell walls of the Phaffia rhodozyma have been developed.

1) A chemical method comprising i) destroying and hydrolyzing cell walls using acid, and ii) neutrolyzing and extracting the astaxanthin has been disclosed in Bacteriol. Rev., 39. 197-231(1975) ; U.S. Pat. No. 5,210,186 and EP 0 553 814 A1.

2) A physical method comprising i) destroying cell walls using French pressure, Braun homogenizer or Microfluidiger, and ii) extracting astaxanthin with solvent has been disclosed in Enzyme Microb. Technol., 8. 194-203(1986); J. Appl. Bacteriology 70. 181-191(1991).

3) A biochemical method comprising the step destroying cell walls using digestion enzyme, such as cellulase, hemicellulase and pectinase has been disclosed in Appl. and Environ. Microbiol. 35(6). 1155-1159(1978).

However, any of above disclosed methods can not afford the commercially available extracting method for astaxanthin, due to the destruction of astaxanthin pigment during the process and the low yield of astaxanthin. Further, the direct extraction method of astaxanthin using the solvent, such as ethanol or acetone, also cannot be commercialized due to its high cost and low yield.

To solve above problems, the inventors developed a microwave treatment method to destroy cell walls for efficient extraction of pigment and also designed the vessel and the tube for microwave irradiation. Therefore, the inventors accomplished effective and cost efficient method for obtaining astaxanthin pigment.

### DISCLOSURE OF INVENTION

The object of the present invention is to provide a process for extracting astaxanthin pigment from the yeast cells of Phaffia rhodozyma comprising the steps of i) cultivating yeast cells, ii) suspending cultivated yeast cells with water iii) treating culture suspension with microwave to destroy the cell walls and microbodies, and iv) drying obtained material containing astaxanthin pigment.

The another object of the present invention is to provide a process for extracting astaxanthin pigment from the yeast cells of Phaffia rhodozyma comprising the steps of i) cultivating yeast cells, ii) suspending cultivated yeast cells with water iii) treating culture suspension with microwave to destroy the cell walls and microbodies, and iv) extracting astaxanthin pigment using the solvent selected from the group consisting of ethanol, methanol, acetone and mixture of them.

The microwave treatment is selected from the continuous process in which the culture suspension is passed through Teflon tube, and the fixed process in which the culture suspension is laid on the Teflon extraction vessel, for irradiation of microwave 50˜1000 watts at the frequency of 900˜930 or 2400˜2500 MHz.

The present invention also provide a process for extracting astaxanthin pigment from the yeast cells of Phaffia rhodozyma, further comprising the pigment separation step, in which the obtained pigment is concentrated at reduced pressure using rotary vacuum evaporator.

The yield of astaxanthin pigment is 10˜95 wt % from total carotenoid contained in the yeast cells of Phaffia rhodozyma, and the purity of astaxanthin pigment is 50˜95 wt % of obtained material.

The amount of extraction solvent is 5˜20 vol. part compared to 1 vol. part of suspension, or 5˜10 (vol./wt) part compared to 1 wt part of dried content of the yeast cells of Phaffia rhodozyma.

The further object of the present invention is to provide a method for using astaxanthin pigment as cosmetics, animal feeds or food additives.

### BRIEF DESCRIPTION OF DRAWINGS

FIG. 1 shows a schematic view of continuous microwave treatment system using the Teflon tube to the suspension according to the present invention.

FIG. 2 shows a schematic view of fixed microwave treatment system using the Teflon extraction vessel to the suspension according to the present invention.

FIG. 3 shows an optical microscope (×1,000) photo of Phaffia rhodozyma without microwave treatment (A); Phaffia rhodozyma with microwave treatment (B) after cultivating it for 5 days according to the present invention.

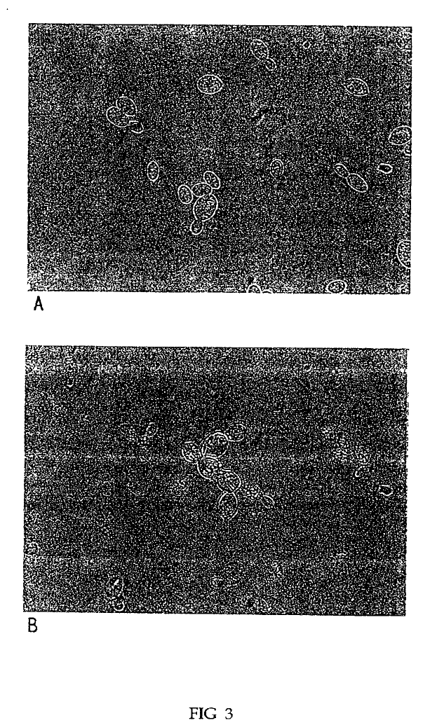

FIG. 4 shows HPLC data of obtained extract followed by i) cultivation of Phaffia rhodozyma ii) microwave treatment, and iii) ethanol extraction according to the present invention.

### BEST MODE FOR CARRYING OUT THE INVENTION

The principle of microwave treatment can be explained as follows.

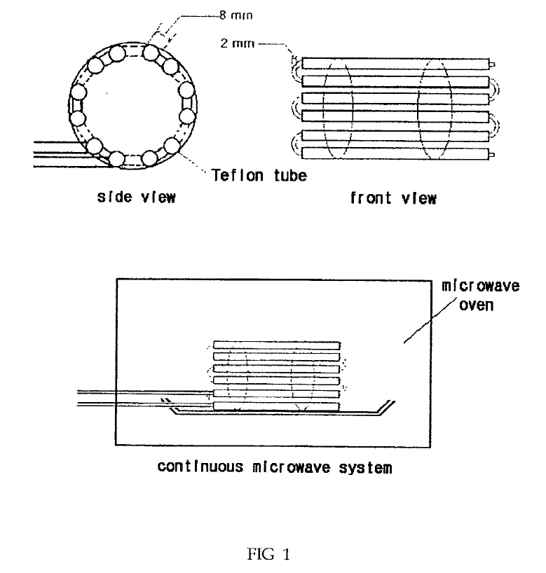

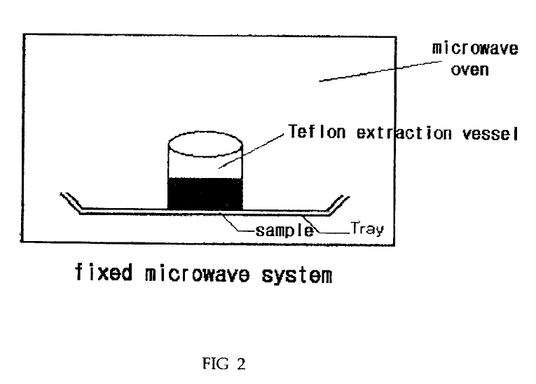

When the microwaves are irradiated into the Phaffia rhodozyma according to the methods shown in FIG. 1 or FIG. 2, the free water and other dipoles in the cells are rotated according to the electric field alternation, which converts microwave energy into thermal energy. Then, the cell walls and microbodies (nucleus, mitochondria, Golgi apparatus) in the cells are destroyed according to the elevation of internal pressure followed by internal heating. Therefore, the microwave treatment enables the extraction of pigment without physical destruction of cell walls. Further, the extraction by organic solvent can be easily accomplished by the microwave treatment, because organic solvent can be easily diffused into the cells.

Therefore, the inventors adopt a microwave treatment method and apply it to the extraction of astaxanthin pigment, which enables the destruction of hard and thick cell walls of Phaffia rhodozyma chiefly composed of α-glucan, especially, α-1,3-glucan.

Further, the inventors design the microwave treatment systems for extracting astaxanthin effectively. One is microwave treatment system using the Teflon tube as shown in FIG. 1 and another is fixed microwave treatment system using the Teflon extraction vessel as shown in FIG. 2.

As shown in FIG. 1, Teflon tube is inserted into the microwave oven and the length and diameter of Teflon tube are designed according to the microwave holding time and microwave treatment capacity. The cultivated suspension is passed through the tube using Variable speed peristaltic pump (Model: AS-90361, Won Corporation, Korea).

As shown in FIG. 2, Teflon extraction vessel is laid on the center of microwave oven and the amount of cultivated suspension is put on the vessel in microwave oven. In this fixed system, Teflon tube and pump are not required.

The output of microwave is in the range of 50˜1,000 watts at the frequency of 916 or 2450 MHz. The microwaves are irradiated into Teflon extraction vessel or Teflon tube for 10˜500 seconds.

The present invention will be more specifically explained by the following examples. However, it should be understood that the examples are intended to illustrate but not in any manner to limit the scope of the present invention.

### EXAMPLES

### Example 1

### Pigment Extraction Yield Test According to Continuous or Fixed Microwave Treatment System

The extraction yield of astaxanthin was measured under microwave illumination according to the conditions; microwave output 50˜1,000 watts, at frequency of 916 or 2450 MHz, for 10˜500 seconds. Cultivated yeast cells (concentration: 10 g/L) were treated with microwave by i) continuous microwave treatment system using the Teflon tube as shown in FIG. 1 and ii) fixed microwave treatment system using the Teflon extraction vessel as shown in FIG. 2. 10 vol. part of ethanol was added to 1 vol. part of cultivated yeast suspension. After extraction at 30° C. for 24 hours, the extract was laid at −20° C. for more than 30 minutes to remove the lipid. After centrifuging obtained extract using centrifuger (10,000×g), the astaxanthin extract was obtained by concentrating the supernatant at reduced pressure. To measure the extraction yield of pigment, absorbance was measured adding acetone by UV/VIS Spectrophotometer Biochrom 4060, Pharmacia, German at 478 nm.

Control group was prepared by following methods. 1 ml of preheated (55° C.) stock solution of dimethylsulfoxide (DMSO) was added to the cultivated yeast cells. Then, the mixture was vigorously agitated in order to fully destroy the yeast cells. To extract the pigment, 1 ml of acetone, 1 ml of petroleum ether and 1 ml of sodium chloride (20%) were added in this order. On storaging in the refrigerator, the solution was centrifuged at 10,000×g for 5 minutes. After obtaining petroleum ether layer in which the pigment was dissolved, the petroleum ether was evaporated at reduced pressure. 1 ml of acetone was added to obtained material and the absorbance was measured at 478 nm for confirming the pigment extract.

The pigment extraction yield was calculated by following equation.

Pigment extraction yield (%)=B/A×100

A: the absorbance of control group after destroying cell walls using DMSO at 478 nm;

B: the absorbance of soluble matter by organic solvent in experimental group after irradiation of microwave at 478 nm

The pigment destruction rate was calculated by following equation.

The pigment destruction rate={A−(B+C)}/A×100

A: the absorbance of control group after destroying cell walls using DMSO at 478 nm;

B: the absorbance of soluble matter by organic solvent in experimental group after irradiation of microwave at 478 nm

C: the absorbance of insoluble matter by organic solvent in experimental group after irradiation of microwave at 478 nm

Table 1 shows the result of pigment extraction yield and pigment destruction rate according to the variation of frequency, time and output. All results are mean values of three times experiments.

TABLE 1

| Frequency | Irradiation time | Output (watt) | | | |
|---|---|---|---|---|---|
| (MHz) | (sec) | 50 | 100 | 500 | 1,000 |
| 916 | 10 | 12(2) | 14(2) | 18(2) | 22(3) |
| | 30 | 19(2) | 23(3) | 54(4) | 52(5) |
| | 60 | 51(3) | 53(5) | 87(5) | 88(7) |
| | 120 | 51(5) | 57(5) | 86(7) | 85(11) |
| | 240 | 53(9) | 69(7) | 84(9) | 83(12) |
| | 500 | 55(6) | 70(9) | 82(12) | 81(13) |
| 2450 | 10 | 13(2) | 15(2) | 19(2) | 23(3) |
| | 30 | 25(5) | 37(5) | 53(4) | 55(5) |
| | 60 | 52(4) | 79(6) | 95(4) | 90(4) |
| | 120 | 51(6) | 77(7) | 90(7) | 88(9) |
| | 240 | 54(7) | 75(8) | 87(7) | 84(11) |
| | 500 | 61(6) | 78(8) | 85(10) | 80(12) |

( ): pigment destruction rate unit: %

### Example 2

### Pigment Extraction Yield Test According to Extraction Solvent and Time After Microwave Irradiation

According to the method in Example 1, frequency 2450 MHz; output 500 watts; and irradiation time 60 seconds were adjusted for microwave treatment. The cultivation suspension has cell density of 10 g/L. The amount of extraction solvent was 10 vol. part compared to 1 vol. part of cultivated suspension. The extraction temperature was at 30° C. The astaxanthin extract was obtained by using rotary vacuum evaporator followed by agitation in the dark room. After adding acetone, the absorbance of pigment extract was measured at 478 nm. Finally, pigment extraction yield was measured using UV/VIS Spectrophotometer Biochrom 4060.

Table 2 shows the result of pigment extraction yield and pigment destruction rate according to the variation of solvent and time. All results are mean values of three times experiments.

TABLE 2

| Extration | Extraction time (hour) | | | | |
|---|---|---|---|---|---|
| solvent | 0 | 6 | 12 | 18 | 24 |
| Control | 2(1) | 8(2) | 11(2) | 13(3) | 15(4) |
| Ethanol | 25(2) | 57(3) | 79(3) | 90(4) | 95(4) |
| Methanol | 27(2) | 63(4) | 81(5) | 82(7) | 82(8) |
| Acetone | 8(2) | 52(5) | 63(6) | 75(6) | 77(8) |

( ): pigment destruction rate unit: %
*Control group shows the absorbance extracted by ethanol from the cultivated suspension without microwave treatment

### Example 3

### Pigment Extraction Yield Test According to Cell Density of Cell Cultivation Suspension at the Time of Microwave Irradiataion

The experiment was carried out as the same manner of Example 2 except that cell densities of cell cultivation suspensions were varied. The cell density of cell cultivation suspension was evaluated by the dried weight of yeast cells per liter. Finally, pigment extraction yield and pigment destruction rate were measured using UV/VIS Spectrophotometer Biochrom 4060.

Table 3 shows the result of pigment extraction yield and pigment destruction rate according to cell density of cell cultivation suspension at the time of microwave irradiation. All results are mean values of three times experiments.

TABLE 3

| cell density (g/L) | Extraction yield (%) |
|---|---|
| 5 | 94(3) |
| 10 | 95(4) |
| 50 | 91(7) |
| 100 | 86(9) |
| 200 | 90(7) |

( ): pigment destruction rate unit: %

### Example 4

### Pigment Extraction Yield Test According to Extraction Temperature and Time After Microwave Irradiation

The experiment was carried out as the same manner of Example 2 except that the extraction temperatures were varied. Finally, pigment extraction yield was measured using UV/VIS Spectrophotometer Biochrom 4060.

Table 4 shows the result of pigment extraction yield and pigment destruction rate according to the variation of extraction temperature and time. All results are mean values of three times experiments.

TABLE 4

| Extraction | Extraction time (hour) | | | | | |
|---|---|---|---|---|---|---|
| temparature | 0 | 6 | 12 | 18 | 24 | 48 |
| 20 | 25(2) | 32(2) | 55(2) | 56(3) | 56(3) | 55(5) |
| 30 | | 57(3) | 79(3) | 90(4) | 95(4) | 89(7) |
| 40 | | 57(3) | 76(3) | 79(5) | 90(6) | 85(9) |
| 50 | | 58(4) | 77(7) | 80(9) | 82(11) | 76(12) |
| 80 | | 62(8) | 69(8) | 74(12) | 75(13) | 70(13) |

( ): pigment destruction rate unit: %

### Example 5

### Pigment Extraction Yield Test According to Extraction Solvent Volume After Microwave Irradiation

The experiment was carried out as the same manner of Example 2 except that the extraction solvent volumes were varied. The extraction time was 24 hours and extraction temperature was 30° C. Finally, pigment extraction yield was measured using UV/VIS Spectrophotometer Biochrom 4060.

Table 5 shows the result of pigment extraction yield and pigment destruction rate according to the variation of extraction solvent volume. All results are mean values of three times experiments.

TABLE 5

| Ethanol:Suspension (V/V) | Extraction yield (%) |
|---|---|
| 1:1 | 10(5) |
| 2:1 | 39(6) |
| 5:1 | 67(7) |
| 10:1 | 95(4) |
| 20:1 | 94(4) |

( ): pigment destruction rate unit: %

### Example 6

### Analysis for Measuring the Astaxanthin Contents in Extracted Pigment

The total carotenoid contents were measured according to known method. 1 ml of cultivated suspension was centrifuged and the precipitate was washed with steriled water twice. Then, 1 ml of preheated (55° C.) stock solution of dimethylsulfoxide (DMSO) was added. Then, the mixture was vigorously agitated in order to fully destroy the yeast cells.

To extract the pigment, 1 ml of acetone, 1 ml of petroleum ether and 1 ml of sodium chloride (20%) were added in this order. On storaging in the refrigerator, the solution was centrifuged at 10,000×g for 5 minutes. The absorbance of the supernatant was measured at 474 nm.

Using 1 ml of pigment extract solution in Example 5, the solvent was evaporated at reduced pressure. 1 ml of petroleum ether was added and the absorbance was measured at 474 nm. The extraction yield of pigment was measured by comparing the amount of pigment extracted by dimethylsulfoxide (100%).

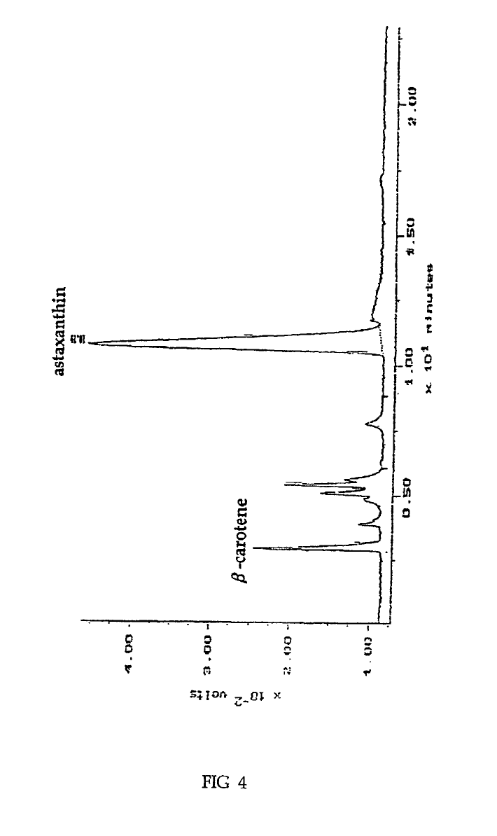

Total carotenoid contents were measured by using following equation which contains 1% extinction coefficient (2,100) and dried content of cells (Appl. and Environ. Microbiol., 55. 116-124(1989)). From ethanol extract, the contents of astaxanthin pigment was measured after evaporating extraction solvent at vacuum and reduced pressure. Then, obtained astaxanthin pigment was dissolved in chloroform before analyzing HPLC (Waters 486, USA). The HPLC data was shown in FIG. 4. Detailed analytical conditions were i) mobile phase=n-hexane/acetone (8:2); ii) stationary phase=silica (4.0×250 mm); iii) solvent=chloroform; iv) flow rate=1 ml/min, v) wave length=476 nm; vi) standard=astaxanthin (Sigma, 98% up) dissolved in chloroform. Then, the contents of astaxanthin were measured according to the standard quantification curve which was prepared by the measurement of standard concentration of astaxanthin.

Total carotenoid contents (mg/g yeast cell dry weight)=(A×M×100)/(21×D)

A: absorbance of pigment at 474 nm;

M: amount of solvent used for extraction (ml);

D: dry weight of Phaffia rhodozyma cells;

21: 1% of extinction coefficient from the weight of cells=2,100

The contents of total carotenoid after microwave irradiation using ethanol solvent and the contents of astaxanthin were measured. The result as shown in Table 6 was mean values of three times experiments.

TABLE 6

| | Extraction | | | | | |
|---|---|---|---|---|---|---|
| | Pigment | | | | | |
| | extraction after | | | | | |
| | DMSO destruction | | | | | |
| | Total | | Pigment extraction after microwave irradiation | | | |
| | carotenoid | Extraction | Total | Extraction | Astaxanthin | Extraction |
| method | (mg/g | yield | carotenoid | yield | (mg/g | yield |
| Cells | yeast) | (%) | (mg/g yeast) | (%) | yeast) | (%) |
| Phaffia rhodozyma | 4.27 | 100 | 4.06 | 95 | 3.65 | 90 |

*The astaxanthin content was measured from the extract after irradiation microwave

### Reference Example

### The Production of Phaffia rhodozyma Dried Product

The example of producing Phaffia rhodozyma dried product was as follows.

After irradiating microwaves to the cultivated Phaffia rhodozyma suspension as the same manner in Example 2, the cells were obtained after continuous flow centrifuges (7,000×g). The obtained cells were washed twice using clean water and the content of water made to be less than 10%. Using spray dryers, vacuum drum dryers or trays dryers, the Phaffia rhodozyma dried product was manufactured.

What is claimed is:

1. A process for extracting astaxanthin pigment from the yeast cells of Phaffia rhodozyma comprising the steps of
i) cultivating yeast cells;
ii) suspending cultivated yeast cells with water;
iii) treating culture suspension with microwave to destroy the cell walls and microbodies; and
iv) drying obtained material containing astaxanthin pigment.

2. A process for extracting astaxanthin pigment from the yeast cells of Phaffia rhodozyma comprising the steps of
i) cultivating yeast cells;
ii) suspending cultivated yeast cells with water;
iii) treating culture suspension with microwave to destroy the cell walls and microbodies; and
iv) extracting astaxanthin pigment using the solvent selected from the group consisting of ethanol, methanol, acetone and mixture of them.

3. The process for extracting astaxanthin pigment from the yeast cells of Phaffia rhodozyma according to claim 1 or claim 2, wherein the microwave treatment is selected from the continuous process in which the culture suspension is passed through Teflon tube, and the fixed process in which the culture suspension is laid on the Teflon extraction vessel, for irradiation of microwave 50˜1000 watts at the frequency of 900˜930 or 2400˜2500 MHz.

4. The process for extracting astaxanthin pigment from the yeast cells of Phaffia rhodozyma according to claim 2, wherein further comprising the pigment separation step in which the obtained pigment is concentrated at reduced pressure using rotary vacuum evaporator.

5. The process for extracting astaxanthin pigment from the yeast cells of Phaffia rhodozyma according to claim 1 or claim 2, wherein the yield of astaxanthin pigment is 10˜95 wt % from total carotenoid contained in blue-green algae, and the purity of astaxanthin pigment is 50˜95 wt % of obtained material.

6. The process for extracting astaxanthin pigment from the yeast cells of Phaffia rhodozyma according to claim 1 or claim 2, wherein the amount of extraction solvent is 5˜20 vol part compared to 1 vol part of suspension, or 5˜10 (vol/wt) part compared to 1 wt part of dried content of blue-green algae.

7. Astaxanthin pigment extracted from the yeast cells of Phaffia rhodozyma according to the method in claim 1 or claim 2.

8. A method for using astaxanthin pigment as cosmetics, animal feeds or food additives.
